(19) United States
(12) Patent Application Publication
(10) Pub. No.: US 2010/0152304 A1
(43) Pub. Date: Jun. 17, 2010

(54) PHARMACEUTICAL COMPOSITION COMPRISING PERIPLANETA AMERICANA OR ITS ETHANOL EXTRACT AND A METHOD OF USING THE SAME FOR TREATING INFLAMMATIONS

(76) Inventor: Funeng GENG, Chengdu (CN)

Correspondence Address: LADAS & PARRY LLP, 224 SOUTH MICHIGAN AVENUE, SUITE 1600, CHICAGO, IL, 60604, US

(21) Appl. No.: 12/336,675

(22) Filed: Dec. 17, 2008

(51) Int. Cl. A61K 31/045; A61P 29/00

(52) U.S. Cl. 514/724

(57) ABSTRACT

The present invention relates to Periplaneta Americana or its ethanol extract for the treatment of inflammation. Specifically, said inflammation includes cervical erosion, ulcerative colitis, skin ulcer, mucosal ulcer and postoperative condyloma acuminatum. Treating said disorders by Periplaneta Americana or its ethanol extract has the advantages of effectively reducing bleeding, shortering healing time, significant curative effect, no adverse reactions and low cost. Periplaneta Americana or its ethanol extract provides a new selection for the clinical treatment of cervical erosion, ulcerative colitis and postoperative condyloma acuminatum.

### TECHNICAL FIELD

The present invention relates to Periplaneta americana or its ethanol extract, specifically, to Periplaneta americana or its ethanol extract for the treatment of inflammation (especially for external application). The present invention also relates to a method of using the same to treat inflammations.

### BACKGROUND ART

It is disclosed in National Drug Standards published by State Drug Administration (issued on Dec. 11, 2000) that Kangfuxin Ye is a solution prepared from ethanol extract of the dry body of Periplaneta Americana. In this standard, description, check, assays, functions and indications, specification, storage and the like, as well as quality specification of Kangfuxin Ye are disclosed. Its functions and indications are promoting blood circulation, nourishing yin and promoting granulation. When taken orally, it is used for blood stasis, stomach pain and gastrorrhagia, stomach/duodenum ulcer, moreover, it is used as adjuvant therapies for yin deficiency and pulmonary consumption as well as pulmonary tuberculosis. When applied externally, it is used in the wound surfaces of incised wound, external injury, ulcer, fistula, burn, scald and bedsore.

Cervical erosion is a common and frequently-occurring disease in women of childbearing age. The symptoms of which are profuse leukorrhea, sour waist, contact bleeding, etc. It disturbs normal work and life. At present, it is usually treated by pome light or microwave. Although such treatments can achieve certain therapeutic effects, both treatments exist questions of different extent of increased vaginal discharge, bleeding in decrustation period, prolonged wound healing time and the like after operation, for example, after microwave treatment, adverse reactions such as cervical wound surface bleeding, vaginal discharge, secondary infection, cervical stenosis and the like are often found, and it is not allowed for the patients to have tubbing, sexual life and vaginal douching for 56 days after operation, which brings about a greater effect on the patients' life.

Ulcerative colitis (UC), referred to as nonspecific ulcerative colitis, is a inflammatory disease of rectum and colon without definite etiology, the lesions of which are mainly confined to colorectal mucosa and submucosa, thus UC is a colonic and rectal disease whose main manifestations are abdominal pain, diarrhea, and mucous bloody stool. Colonoscopy in combination with biopsy is the most reliable diagnosis method. At present, because UC's etiology and the exact pathogenesis are still not completely elucidated, therapeutic methods and effects reported are different. SASP (salicylazosulfapyridine) or 5-ASA (5-aminosalicylic acid) is the main drug used for the clinical treatment of UC in internal medicine. However, SASP has prominent toxic side effects when used in a larger amount, and 5-ASA is high in price, which are the cause limiting the wide use of SASP and 5-ASA. It is also reported that the treatment of UC by a clyster with the mixture of Xilei San and Yunnan Baiyao can obtain certain therapeutic effects, however, the clyster is tedious to be formulated, easy to be contaminated, and has poor patients compliance.

Condyloma acuminatum (short for CA) is a disease infected by human papillomavirus (HPV) through sexual contact. Genital condyloma acuminatum is typically asymptomatic without pain or agony, however, pruritus will occur when the condyloma gets cankered and eroded, moreover, pruritus would result in secondary infection accompanied with pain. The routine therapeutic methods of condyloma acuminatum include laser, freezing, local externally application of drugs, electric cauterization, operation and the like. In addition, drugs such as interferon, thymosin, transfer factor and the like can also be used. Such methods and drugs can render the bodies of the warts disappear rapidly, yet they are not capable of solving fundamentally the problem of replication of papillomavirus, only resulting in temporary disappearance of surface symptoms. In this case, the viruses in vivo cannot be removed clearly, so it is easy for condyloma acuminatum to recur again after recovery. At the same time, the wound surfaces are easy to be infected by bacterium, or to come with mild to severe erosion or bleeding after operation. The erosion manifests as lost of normal epidermis, the exposure of wetted surface, red fundus and margin of skin, as well as more or less of transudate. Bleeding may be more obvious, such as the damage of larger vessels, or may be less obvious, such as little staxis, blood trails and scabs present on gauzes bandaging the wound, underwares and parts thereof; even edema and the like would occur. After operation, preventive measures such as cleaning the wound surfaces with potassium permanganate solution and smearing antibiotics ointment are often taken, however, the effects are not satisfying.

Use of Periplaneta americana or its ethanol extract for the treatment of cervical erosion, ulcerative colitis and postoperative condyloma acuminatum have not been reported.

### DISCLOSURE OF THE INVENTION

In one aspect, the present invention provides an ethanol extract of Periplaneta americana.

In another aspect, the present invention provides a pharmaceutical composition for the treatment of inflammation, comprising an effective amount of Periplaneta Americana or its ethanol extract as active ingredients, and optionally a pharmaceutically acceptable adjuvant or auxiliary component, wherein, said extract of Periplaneta Americana is prepared with 75% to 95% ethanol.

In yet another aspect, said pharmaceutical composition is a certain pharmaceutical preparation, especially a pharmaceutical preparation used for external application.

Preferably, the extract of Periplaneta americana is prepared with 95% ethanol.

Wherein, said medicament used for the treatment of inflammation is a drug used for the treatment of cervical erosion, each dosage unit contains Periplaneta americana or its ethanol extract equivalent to 2 g˜10 g of raw material of Periplaneta Americana.

Said medicament used for the treatment of inflammation is a drug used for the treatment of ulcerative colitis, each dosage unit contains Periplaneta americana or its ethanol extract equivalent to 2 g˜10 g of raw material of Periplaneta Americana.

Said medicament used for the treatment of inflammation is a drug used for the treatment of postoperative condyloma acuminatum, each dosage unit contains Periplaneta americana or its ethanol extract equivalent to 2 g˜10 g of raw material of Periplaneta Americana.

Where the above three inflammations are treated, it is preferable that each dosage unit contains 5 g of raw material of Periplaneta Americana.

Said each dosage unit according to the present invention refers to the one-time dose of the formulation prepared with ethanol extract of Periplaneta Americana, i.e., one dosage unit is used for each time. The one-time dose should be determined by actual situation, such as absorption amount of cotton balls and gauzes, as well as smearing area.

The effective dosage of raw material of Periplaneta Americana in the present invention is determined by the conversion of clinical dosage of ethanol extract of Periplaneta Americana. It can be deduced according to ethanol extract of Periplaneta Americana prepared in example 1 that every 10000 ml contains 1000 g of ethanol extract of Periplaneta Americana, then it can be further deduced that the dosage of Periplaneta Americana or ethanol extract thereof according to the present invention applied externally for the treatment of cervical erosion, ulcerative colitis and postoperative condyloma acuminatum equivalents to 2˜10 g of raw material of Periplaneta Americana, preferably 5 g.

The present invention also provides a pharmaceutical composition useful for the treatment of inflammation, which is a pharmaceutical preparation made of effective amount of Periplaneta Americana or its' ethanol extract as active ingredients plus a pharmaceutical acceptable adjuvant or auxiliary component.

Wherein, said pharmaceutical preparation is a formulation for external application. Specially, said pharmaceutical preparation includes liniment, lotion, ointment, suppository, spray and gel. Of course, the pharmaceutical preparation in the present invention can be a preparation taken by orally.

Ethanol extract of Periplaneta Americana can be selected for the preparation of the above dosage forms depending on specific disorders and patients' need. For example, suppository can be selected to be placed into vaginae for the treatment of cervical erosion, and ointment can be selected for the treatment of postoperative condyloma acuminatum.

Periplaneta Americana or its' ethanol extract have the following functions: promoting vascular proliferation, eliminating inflammatory edema, improving the local microcirculation, accelerating the repair of lesion tissue, enhancing immunity of the body, activating nonspecific immune cells and resisting pathogenic materials through direct phagocytosis. The present invention employs Periplaneta Americana or its' ethanol extract for the treatment of cervical erosion, ulcerative colitis and postoperative condyloma acuminatum, which has the advantages of effectively reducing bleeding, shorter healing time, low cost, significant curative effect and no adverse reactions. The present invention provides a new selection for the clinical treatment of cervical erosion, ulcerative colitis and postoperative condyloma acuminatum.

It is apparent that those skilled can also make other various modifications, alternations and change to the present invention according to general technical knowledge and routine means in the art without departing from the basic technical spirits of the present invention.

The detailed description of the present invention is illustrated by the following specific embodiments by way of examples. However, it should not be understood that the scope of the above subjects of the present invention are restricted in the following examples. All the achieved techniques based on the above contents of the present invention fall in the scope of the present invention.

### SPECIFIC MODELS FOR CARRYING OUT THE INVENTION

### EXAMPLE 1

### Preparation of Ethanol Extract of Periplaneta Americana of the Present Invention

Dried Periplaneta Americana was crushed roughly. 4000 g water was added to every 1000 g crude powder. After being soaked for 1 hour, the resultant mixture was extracted at about 70° C. for three times. The first time is for 8 hours; the second time is for 6 hours with adding 3000 g water; the third time is for 4 hours with adding 3000 g water. The three extracts were combined, filtered, and the filtrate was concentrated to a relative density of 1.10˜1.20(70° C.), then 95% ethanol (3000 g) was added. The resultant mixture was kept at 70° C. and stirred for 30 minutes. After standing for 12 hours, the oil and fat of the upper layer was discarded and the solution of the lower layer was filtered. Ethanol was recovered from the filtrate, then the filtrate was concentrated under reduced pressure to a relative density of 1.20˜1.25(70° C.), 500 ml of glycerol was added, and the mixture was stirred thoroughly, filtered, and added purified water to 10000 ml.

### EXAMPLE 2

### Preparation of Ethanol Extract of Periplaneta Americana of the Present Invention

Dried Periplaneta Americana was crushed roughly. 4000 g water was added to every 1000 g crude powder. After being soaked for 1 hour, the resultant mixture was extracted at about 70° C. for three times. The first time is for 8 hours; the second time is for 6 hours with adding 3000 g water; the third time is for 4 hours with adding 3000 g water. The three extracts were combined, filtered, and the filtrate was concentrated to a relative density of 1.10˜1.20(70° C.), then 75% ethanol (3000 g) was added. The resultant mixture was kept at 70° C. and stirred for 30 minutes. After standing for 12 hours, the oil and fat of the upper layer was discarded and the solution of the lower layer was filtered. Ethanol was recovered from the filtrate, then the filtrate was concentrated under reduced pressure to a relative density of 1.20˜1.25(70° C.), 500 ml of glycerol was added, and the mixture was stirred thoroughly, filtered, and added purified water to 10000 ml.

### EXAMPLE 3

### Preparation of Ethanol Extract of Periplaneta Americana of the Present Invention

Dried Periplaneta Americana was crushed roughly. 4000 g water was added to every 1000 g crude powder. After being soaked for 1 hour, the resultant mixture was extracted at about 70° C. for three times. The first time is for 8 hours; the second time is for 6 hours with adding 3000 g water; the third time is for 4 hours with adding 3000 g water. The three extracts were combined, filtered, and the filtrate was concentrated to a relative density of 1.10˜1.20(70° C.), then 85% ethanol (3000 g) was added. The resultant mixture was kept at 70° C. and stirred for 30 minutes. After standing for 12 hours, the oil and fat of the upper layer was discarded and the solution of the lower layer was filtered. Ethanol was recovered from the filtrate, then the filtrate was concentrated under reduced pressure to a relative density of 1.20˜1.25(70° C.), 500 ml of glycerol was added, and the mixture was stirred thoroughly, filtered, and added purified water to 10000 ml.

### EXAMPLE 4

### Preparation of Formulations of the Present Invention

1. lotion: what obtained in examples 1-3 are lotions.

2. suppository: Repeat the method as recited in examples 1˜3 from the beginning to “Ethanol was recovered from the filtrate”, then the filtrate was concentrated to dry extract, subsequently, 10 g of borax and 10 g of borneol were added, smashed, and porphyrized, and 500 g of S-40 was heated in a water bath until it melt, then porphyrized fine powder of the dry extract was added to the above matrix and grinded uniformly, at last, the resultant mixture was kept warm and filled into the suppository mold. Each suppository contains about 2˜10 g of raw material of Periplaneta Americana.

3. ointment: Repeat the method as recited in examples 1˜3 from the beginning to “Ethanol was recovered from the filtrate”, then the filtrate was concentrated to dry extract, subsequently, 10 g of borax and 10 g of borneol were added, smashed and porphyrized; 400 g of polyethylene glycol 3350 was mixed with 600 g of polyethylene glycol 400 and the resultant mixture was heated to 65° C. in a water bath, cooled under stirring to obtain the matrix; then porphyrized fine powder of the dry extract was added to the above condensed matrix and mixed uniformly. Every gram of ointment contains about 2˜10 g of raw material of Periplaneta Americana.

The following clinical pharmacodynamic tests are used to demonstrate the beneficial effects of Periplaneta Americana and its ethanol extract for the treatment of cervical erosion, ulcerative colitis and postoperative condyloma acuminatum.

### TRIAL EXAMPLE 1

### Clinical Trial of Ethanol Extract of Periplaneta Americana for the Treatment of Cervical Erosion

A. The Treatment of Cervical Erosion with Ethanol Extract of Periplaneta Americana in Combination with Pome Light

1.1 Data: 98 outpatients with cervical erosion visiting during the period from September, 2003 to April, 2004 were all married, whose ages ranged from 25 to 35. Before operation, all the patients were subjected to examination of vaginal secretion to exclude an acute inflammation and were subjected to cervical scraping smear to exclude a canceration.

1.2 Diagnostic criteria: Classification was made according to reference document^{[1]}. All the patients were randomized into two groups: 50 patients in treatment group (ethanol extract of Periplaneta Americana prepared in example 1 (extract of example 1 for short) in combination with pome light therapy) and 48 patients in control group (pome light therapy only).

1.3 Methods: POME-III pome light therapeutic device (Dalian Pome Instrument & Equipment Co., Ltd.) was adopted for the treatment. Treatment group was subjected to routine Pome light therapy, followed by inserting a cotton ball with a tail soaked with extract of example 1 (the cotton ball imbibed about 10˜30 ml extract) into the vaginae and kept there for 6 hours, then taken out. Administration was repeated on 1, 2, 3, and 4 weeks after operation, respectively. For treatment group, pome light therapy was as the same as the above, after operation, a iodophor cotton ball was applied to pack the vaginae. Healing of wound surface, vaginal discharge, and vaginal bleeding were observed and recorded upon follow-up. Summarization was made upon 8 weeks after the operation.

1.4 Evaluation standards of therapeutic effects: “cure” manifests as smooth unerine neck and disappear of erosion surfaces; “markedly effective” manifests as an improvement to degree I or erosion area reduced by >50%; “effective” manifests as an improvement to less than degree I or erosion area reduced by <50%; “ineffective” manifests as no change of erosion area or development of erosion surface.

1.5 Statistic methods: t-test and χ² test were adopted.

2. Results

2.1 Comparison of therapeutic effects between two groups: therapeutic effects were evaluated upon 8 weeks after operation, seen from table 1. For treatment group, there appeared epidermis covering erosion surfaces 2 weeks after operation, epidermises of unerine neck in 50 cases were substantially repaired 4 weeks after operation. For control group, the repair of epidermis of unerine neck was relatively and apparently slow, epidermises of unerine neck in 33.33% cases were totally repaired 8 weeks after operation.

TABLE 1
comparison of therapeutic effects of cervical erosion in 98 cases
case number (%)

| | | | | | total |
|---|---|---|---|---|---|
| | | markedly | | | effective |
| group | cure | effective | effective | ineffective | rate/% |
| treatment | 30 (60) | 12 (24) | 4 (8) | 4 (8) | 92 |
| group | | | | | |
| control | 16 (33.33) | 14 (29.17) | 10 (20.83) | 8 (16.67) | 83.33 |
| group | | | | | |

χ² test, P < 0.05, the difference was significant.

2.2 Comparison of the amount of vaginal discharge: for treatment group, the amount of vaginal discharge after operation reduced markedly, and none of which was larger than the amount of menstrual discharge, time of vaginal discharge was 7˜10 days; for control group, the amount of vaginal discharge was larger than that of menstrual discharge in 5 cases, time of vaginal discharge lasted for 10˜20 days.

2.3 Comparison of bleeding in decrustation period: for treatment group, blood scabs formed were thinner and easy to drop off, during dropping off, the amount of vaginal bleeding was little and none of which was larger than the amount of menstrual discharge, mean time of bleeding was 6.02±2.14 days. For control group, the amount of bleeding in 2 cases was larger than that of menstrual discharge, and mean time of bleeding was 11.82±4.31 days. T test showed that the difference was significant (P<0.05) and time of vaginal bleeding in treatment group was reduced markedly.

3. Discussion: treatment of cervical erosion with Pome light only can achieve certain therapy effects. However, adverse reactions are quite prominent. As reported by reference documents, the amount of vaginal discharge after operation was large and this course lasted for 10˜15 days, bleeding in decrustation period lasted for about 10˜15 days. In most situations, the repair of epidermis of unerine neck required 6˜8 weeks, this course was quite long^{[2]} and was basically as the same as Pome light control group in trial example 1. Treatment with ethanol extract of Periplaneta Americana in combination with pome light can make up for the deficiency of the treatment with pome light only. Analyzed from pharmacology, ethanol extract of Periplaneta Americana is rich in polyols and polypeptides (Epidermal Growth Factor, EGF), which can promote epidermal cell growth and granulation tissue proliferation, improve local blood circulation, accelerate desquamate of necrotic tissue on wound surfaces, and expedite the repair of wound surfaces. For treatment group, the total effective rate was 92%, which was higher than that of control group (83.33%)(P<0.05). The epidermis of unerine neck was repaired rapidly and blood scabs was easy to drop off, moreover, time of vaginal bleeding was short and the amount of vaginal bleeding was little. Meanwhile, ethanol extract of Periplaneta Americana also contains mucoaminoglycan (niantang'ansuan), which can enhance phagocytic functions of phagocytes and NK cells, promote secreting interleukin-like, interferon and leukotriene and the like, thereby it can eliminate inflammatory edema. No infection was found in all the cases in both groups. For treatment group, the cervical edema was mild and the amount of vaginal discharge was obviously less than that of control group. During the treatment, no adverse reactions caused by ethanol extract of Periplaneta Americana were found. Treatment of cervical erosion with ethanol extract of Periplaneta Americana in combination with pome light can improve therapeutic effects and reduce adverse reactions. Additionally, for the above treatment, it is easy to operate and no obvious side effects were found.

B. Treatment of Cervical Erosion with Ethanol Extract of Periplaneta Americana in Combination with Microwave

1. Data and Methods

1.1 General data: 216 patients confirmed as cervical erosion in gynecological department during the period from January, 2004 to November, 2004 were subjected to routine gynecological examination to exclude an acute inflammation of internal and external genitalia, and were subjected to cervical scraping smear to exclude a canceration or other specific inflammations, such as tuberculosis and the like. All the patients were randomized into two groups: 106 patients in group A, which were subjected to local application of extract of example 1 in cervix, and 110 patients in group B, which were subjected to therapy of unerine neck by microwave. The mean age of the two groups were 31.5 and 32.2, respectively. There were no significant differences in age, classification and degree of cervical erosion between the two groups (P>0.05).

1.2 Treatment methods: For group A, extract of example 1 was used. Unerine neck was exposed using vaginal speculum, then unerine neck and vaginal secretion were washed with normal saline, thereafter, a sterilized medical gauze soaked with the extract solution(the gauze imbibed about 10˜30 ml of the extract solution) was applied to the unerine neck locally, dressing change per 24 hours and for 2 weeks. For group B, HSB-B multi-functional microwave operation therapeutic instrument (Nanjing Huabei electronic medical equipment Co., Ltd.) was used. The operation was performed 3˜7 days after the menstrual discharge was clean. During 3 days before operation, sexual life was prohibited. Unerine neck was exposed using vaginal speculum, then unerine neck and vaginal secretion were washed with normal saline, thereafter, unerine neck was radiated evenly from the inside to outside annularly at a powder of 50˜60 w, the radiation was beyond the wound surfaces 0.5 cm, moreover, suitable time of radiation was indicated by the change of erosion surfaces to grey-white or yellowish.

1.3 Evaluation standards of therapeutic effects: follow-up was made 3 months after treatment to evaluate therapeutic effects. “cure” manifests as smooth unerine neck and disappear of erosion surfaces; “markedly effective” manifests as erosion surface reduced by >50% or an transformation of degree II to degree I, or degree III to degree II; “effective” manifests as erosion area reduced by <50% with an obvious reduction yet improvement less than degree I or no obvious reduction of erosion area yet with a transformation of granular type to single type, or nipple type to granular type; “ineffective” manifests as no change of erosion area or development of erosion surfaces.

1.4 Statistics method: χ² test was adopted.

2 Results

2.1 result of therapeutic effects: the results were seen table 2, χ² test showed no significant difference between the two groups (P>0.05).

TABLE 2
results of therapeutic effects of the two groups case number

| | | | | | | total |
|---|---|---|---|---|---|---|
| | case | | markedly | | | effective |
| group | number | cure | effective | effective | ineffective | rate/% |
| group A | 106 | 756 | 10 | 17 | 4 | 96 |
| group B | 110 | 81 | 9 | 15 | 5 | 95 |

2.2 Adverse reactions: after microwave operation, most of the patients are found with little bleeding of wound surfaces of unerine neck and a large amount of vaginal discharge, wherein 15 cases were found with a large amount of bleeding of wound surfaces of unerine neck, 9 cases were found with acute internal and external genital inflammations, 4 cases were found with cervical stenosis after operation. For group A, no above symptoms and other obvious adverse reactions were found.

3. Discussion: Treatment of cervical erosion with extract of example 1 or microwave has a definite curative effect, there is no significant difference between the therapeutic effects of the two groups. After treatment with microwave, adverse reactions such as bleeding of wound surfaces of unerine neck, virginal discharge, secondary infection, cervical stenosis and the like were often found, additionally, tub bath sexual intercourse and vaginal douching were prohibited for 56 days after operation, which affected the patients' life greatly. Whereas, the treatment for cervical erosion with ethanol extract of Periplaneta Americana has the advantages of short course, safety, high efficacy, convenience, no pain, and easy to be accepted by patients and be extended clinically.

### TRIAL EXAMPLE 2

### Treatment for Ulcerative Colitis with Retention Enema of Ethanol Extract of Periplaneta Americana

1. General data: 40 patients were all confirmed by clinical examination, stool routine and colon examination. Among them, 28 patients were male and 12 female. Their ages ranged from 16 to 70 and mean age was 45. The course of disease ranged from 3 months to 2 years.

Diagnostic criteria: according to the diagnostic criteria prescribed by the national academic conference on chronic and non-infective intestinal disease in 1993, 6 cases were right-sided colitis, 9 cases were pancolitis, 25 cases were left-sided colitis. Classification was made according to the disease degree: 14 cases were mild, 19 cases were moderate, 7 cases were severe.

2. Treatment methods: after hospitalization, patients were given a mixed enema liquid, which is composed of 50 ml of extract of example 1, 100 ml of 0.2% metronidazole solution and 5 mg of dexamethasone, the enema liquid was applied for retention enema of above 1 hour once a night when heated to about 38° C., 20 days as a course. If the retention effect was not satisfactory, 30 ml of 1% procaine solution or 10 ml of 1% lidocaine solution was added to the enema liquid. For severe patients, 200 ml hydrocortisone was administered in combination with intravenous infusion of 5% glucose simultaneously for 5˜7 days.

3. Evaluation Standards of Therapeutic Effects and Results:

3.1 Standards: “near-term cure”: symptoms disappears, no abnormal with mucoma when reexamined under colonoscopy, no recurrence found for 6 months under the situation of drug withdrawal or maintenance drug therapy only; “effective”: symptoms disappears basically, mild inflammatory reactions can be found with mucoma when reexamined under colonoscopy, with congestive edema or partial pseudopolyp formation; “ineffective”: no improvement in symptoms and endoscope examination.

3.2 Results: among 40 cases, 32 cases were cured, 5 cases effective, 3 cases ineffective, and the effective rate is 92.5%, determined according to clinical symptoms and results of colonoscopy. In this trial, most of the patients had experienced regular treatment of 1˜2 courses, the one who had experienced the longest therapy time was a severe patient, whose symptoms were relieved after a 4-course therapy. 3 ineffective cases were all severe patients, two of which were transferred to surgery department due to complicated hemorrhage, one of which was transferred to a superior hospital for therapy due to no obvious change of symptoms after 1 course. Among all the cases, one patient had mild nausea 20 minutes after enema, and this side effect disappeared when metronidazole was displaced by tinidazole. For other patients, no obvious side effects and severe complications were found. The relationship between severity of disease and therapeutic effects was summarized in table 3.

TABLE 3
the relationship between severity of disease and therapeutic effects case

| Group | n | near-term cure | effective | inineffective |
|---|---|---|---|---|
| Mild | 14 | 12 | 2 | 0 |
| moderate | 19 | 15 | 4 | 0 |
| Severe | 7 | 1 | 3 | 3 |

4. Discussion: In this trial, all the 40 patients were confirmed by colonoscope, wherein 25 cases were left-sided colitis(62.5%), 9 cases were pancolitis (22.5%) and 6 cases were right-sided colitis(15%). This disease often involved in distal colon, therefore, treatment with drug retention enema can render the drug contact directly with lesion site, thereby obtaining better therapeutic effects and reducing side effects obviously compared with systemic administration. The effective rate of this trial was 92.5%, only 1 case had mild side effects, therefore, local administration had a prominent advantage. The present invention applies retention edema of ethanol extract of Periplaneta Americana, metronidazole plus hormones once a night, which can inhibit inflammation and immune response, relieve toxic symptoms, and promote the repair of mucosa, thus it achieves better therapeutic effects. Moreover, this edema liquid has little side effects, so it is effective and safe, low in price, and easy to be extended clinically.

### TRIAL EXAMPLE 3

### Treatment of Condyloma Acuminatum with Ethanol Extract of Periplaneta Americana

1. Data and Methods

1.1 Case selection: all the cases were out-patients with venereal disease who were primarily diagnosed to be condyloma acuminatum by clinicians and confirmed via 5% Acetowhite test. The patients who had more than 3 warts each smaller than housebeen (diameter not larger than 1 cm) were determined as observation cases. Among 62 cases who had finished the observation, 43 cases were male, and 19 cases female; their ages ranged from 22 to 43 years, mean age 27.5 years; the course of disease ranged from 2 to 5 months. All the patients were randomized into treatment group (30 cases) and control group (32 cases). The two groups were comparable in sex, age, course, as well as number and size of lesions.

1.2 Treatment methods: all the patients were performed high-frequency electrotome under topical anaesthesia to remove warts, after operation, treatment was performed to the wound surfaces. For treatment group, extract of example 1 was smeared after operation; when returned home, the patients used sterilized cotton ball soaked with extract of example 1 to smear on the wound surfaces (the administration amount of extract was about 50 ml˜100 ml), 20 minutes for each time, bid, till the wound surface healed. Typically, the patients returned for a visit per 3 days to check the healing of wound surfaces. For control group, administration method was as the same as treatment group, except that the medicine solution used was gentamicin injection solution.

2 Results: mean healing time was 6.07±1.55 days for treatment group, and 9.13±1.72 days for control group. There was significant difference between the two groups in healing time (P<0.01).

3 Discussion: ethanol extract of Periplaneta Americana had an obvious effect of promoting healing on the wound surfaces of condyloma acuminatum. The edema and exudation of wound surfaces in treatment group after administration was prominently milder than that of control group. No secondary infections were found for both groups. Additionally, according to follow-up for parts of patients, it was also found that the patients with condyloma acuminatum applied with ethanol extract of Periplaneta Americana had a slightly lower recurrence rate than that of control group.

According to the above trials, it can be demonstrated that the treatment of cervical erosion, ulcerative colitis and postoperative condyloma acuminatum by applying ethanol extract of Periplaneta Americana of the present invention has the following advantages: easy to operate, prominent therapeutic effects and no adverse reactions.

### REFERENCE DOCUMENTS

[1] Huaimei Zheng, Obstetrics and Gynecology [M], 3rd, Beijing: People's Health Publishing House, 1987, 253;

[2] Muyu Liu, Zaoxiong He, Observation on the effectiveness of Aopin in combination with Pome for the treatment of cervical erosion, Journal of Practical Obstetrics and Gynecology, 2002, 18(3): 313.

What is claimed is:

1. An ethanol extract of Periplaneta Americana, which is prepared from Periplaneta Americana with ethanol of a concentration from the range of 75% to 95%.

2. The ethanol extract of Periplaneta Americana according to claim 1, which is prepared with 95% ethanol.

3. A pharmaceutical composition for the treatment of inflammation, comprising an effective amount of Periplaneta Americana or its ethanol extract according to claim 1 as active ingredients, and optionally a pharmaceutically acceptable adjuvant or auxiliary component.

4. The pharmaceutical composition according to claim 3, wherein said pharmaceutical composition is a pharmaceutical preparation used for external application.

5. The pharmaceutical composition according to claim 3, wherein said inflammation is cervical erosion.

6. The pharmaceutical composition according to claim 5, wherein said pharmaceutical preparation contains Periplaneta americana or its ethanol extract equivalent to 2 g˜10 g of raw material of Periplaneta Americana per dosage unit.

7. The pharmaceutical composition according to claim 3, wherein said inflammation is ulcerative colitis.

8. The pharmaceutical composition according to claim 7, wherein said pharmaceutical preparation contains Periplaneta americana or its ethanol extract equivalent to 2 g˜10 g of raw material of Periplaneta Americana per dosage unit.

9. The pharmaceutical composition according to claim 3, wherein said inflammation is postoperative condyloma acuminatum.

10. The pharmaceutical composition according to claim 9, wherein said pharmaceutical preparation contains Periplaneta americana or its ethanol extract equivalent to 2 g˜10 g of raw material of Periplaneta Americana per dosage unit.

11. A method used for the treatment of inflammation, comprising administering Periplaneta americana or its ethanol extract according to claim 1 or the pharmaceutical composition according to claim 3 to a subject.

12. The method according to claim 11, wherein said Periplaneta americana or its ethanol extract or the pharmaceutical composition according to claim 3 is used for external application.

13. The method according to claim 11, wherein said inflammation is cervical erosion.

14. The method according to claim 11, wherein said inflammation is ulcerative colitis.

15. The method according to claim 11, wherein said inflammation is postoperative condyloma acuminatum.
